(19) United States
(12) Patent Application Publication
(10) Pub. No.: US 2009/0030289 A1
(43) Pub. Date: Jan. 29, 2009

(54) Biological Information Monitoring System

(75) Inventors: Noritada Katayama, Kanagawa (JP); Nobuaki Kawaguchi, Tokyo (JP); Nobuyoshi Kurosawa, Tokyo (JP)

Correspondence Address: WENDEROTH, LIND & PONACK, L.L.P., 2033 K STREET N. W., SUITE 800, WASHINGTON, DC, 20006-1021, US

(73) Assignee: INTELLECTUAL PROPERTY BANK CORP., Tokyo (JP)

(21) Appl. No.: 11/794,347

(22) PCT Filed: Dec. 27, 2005
(86) PCT No.: PCT/JP2005/023938

§ 371 Date: Jun. 28, 2007

(30) Foreign Application Priority Data
Dec. 28, 2004 (JP) 2004-378390

(51) Int. Cl. A61B 5/02; A61B 5/00; A61B 5/01

(52) U.S. Cl. 600/301; 600/549; 600/485; 600/500

(57) ABSTRACT

The present invention provides a biological information monitoring system capable of determining a physical abnormality at its earliest stage with higher accuracy than conventional methods by detecting biological information at multiple positions of the body of a subject.

The biological information monitoring system according to the invention comprises a plurality of biological information sensor modules to be attached to at least one set of positions of upper and lower bodies, upper and lower extremities, ventral and dorsal surfaces, complementary upper and lower parts and complementary front and back parts of the body of a subject, which biological information sensor modules each incorporate a biological information sensor for detecting the biological information and a wireless-enabled communication means capable of communicating the biological information, in which a determination means for determining an abnormality by comparing the biological information detected by the biological information sensor incorporated in one of the biological information sensor modules itself with another biological information received by the communication means in the other biological information sensor module.

This invention relates to a biological information monitoring system capable of determining the presence or absence of physical abnormality, an effect and excessive extent of sports training workout by detecting biological information such as body surface temperature, pulsebeat, blood pressure, myoelectric potential, sounds such as breath sound, physical motion, inclination and orientation, and graphic data or information as to ambient surroundings.

### BACKGROUND ART

Generally, determination of physical abnormality has been so far carried out while detecting biological information such as body surface temperature, pulsebeat and blood pressure. Typically, a biological information sensor is attached onto the body of a subject being inspected by the subject himself, a doctor or a nurse to detect the physical information. For instance, the body surface temperature is measured by placing a body surface thermometer in the underarm for a few minutes or taking the pulse rate or blood pressure of the subject from one arm of the subject over time for a few minutes.

However, there are cases where the states of the subject being inspected could not thoroughly be grasped only from the biological information passingly measured by the aforementioned inspection method. If the subject being inspected is aware of subjective symptom of abnormality by way of example, the abnormality may not appear during a biologic examination performed by the doctor or nurse, thus to disable accurate diagnosis. Since it is common that arrhythmia and heartbeat abnormality do not always appear during a physical examination, these abnormalities are possibly misidentified in inspecting within a short time due to the psychological state of the subject being inspected or temporal conditions for the inspection.

Hence, there has been a method for continuously detecting the biological information of a subject suspected of having physical abnormalities over a prolonged period of time including sleeping hours.

For instance, there have been recently proposed some methods for determining abnormalities by detecting biological information by means of a small and light-weight biological information sensor attached to the body of the subject and transmitting data thus detected to a data analysis means by wireless. In the conventional method, there have been a certain degree of advance in that a biological information sensor module is reduced in size and weight by incorporating the biological information sensor and the data analysis means therein, so as not to restrain the motion of the subject being inspected.

For instance, a “system for monitoring and alerting human health condition” disclosed in Japanese Patent Application Publication HEI 10-155749(A) can measure physical data such as pulsebeat, motion, sound and body surface temperature of a subject in real time by use of a life sensor attachable to the human body, which is operated in conjunction with an alerting system for giving the physical data to a caregiver on the basis of the information detected by the sensor. The life sensor may be made of a wristwatch type by incorporating a communication means therein. A monitoring center is provided with a communication system for calling for the subject being inspected.

As well, an “abnormal symptom detector and abnormality warning system” disclosed in Japanese Patent Application Publication No. 2000-93398(A) is composed of a skin stretch/shrink detection sensor for detecting the stretching and shrinking of the skin with each pulsebeat and breath and a warning circuit for issuing a warning of abnormality when detecting the abnormality. As the skin stretch/shrink detection sensor, a strain gauge is used. When detecting any abnormality, a telephone communication system is put to practical use.

An “emergency relief system” disclosed in Japanese Patent Application Publication HEI 6-242206(A) is relevant to a system for detecting human abnormality, comprising a wristwatch-type transmitter capable of transmitting faint radio waves, which incorporates or is connected to a sensor for detecting pulsebeat, blood pressure or body surface temperature, a radio relay device with a receiving part for receiving the faint radio waves and a transmitting part for transmitting emergency signals responsive to a specific signal from the receiving part, and a radio station for detecting the position of the radio relay device by receiving the emergency signals.

An “information detector for acquiring physical information” disclosed in Japanese Patent Application Publication No. 2001-353130(A) is featured by incorporating a sensor for detecting the physical information and an output means in a curviform housing case formed in a curviform arch shape as a whole so as to be fitted onto the auricle of a subject being tested. The sensor and output means may sometimes be set in a pendant-like case.

Further, Japanese Patent Application Publication No. 2003-24287(A) proposes a “device for monitoring physical condition” by calculating the physical condition on the basis of outputs from a motion sensor (for detecting at least one of acceleration velocity and angular velocity) and a sphygmographic sensor. Each sensor to be put on the upper limb or lower limb of the subject has a gyro sensor for detecting the angular velocity about the Z-axes perpendicular to the longitudinal direction and transverse direction of the upper limb and lower limb. The monitoring device further has pulse calculating means with denoising means for filtering out noises in the output from the sphygmographic sensor when issuing pulse output from the sensor for detecting the acceleration velocity.

All the conventional measuring devices as described above serves to wirelessly measure various biological information such as pulsebeat, motion, sound, body surface temperature and myoelectric potential, but can make merely local measurements on a part of a human body and cannot detect physical abnormalities at a plurality of parts of the human body by determining an abnormal physical differences at the plurality of parts of the human body.

Then, taking one step further, there has been proposed a “biological information monitoring system” in PCT International Patent Publication No. WO2004/89202, which is featured by attaching sensor to either side of the human body. The proposed system using the plurality of sensors makes it possible to differentiating cerebral infarction and cardiac infarction in their earliest stages by detecting an aberrant difference between measured values, even if each measured value is within the range of normality.

However, the physical abnormalities do not occur only on the right and left sides of the human body. That is, there is a possibility that the physical abnormality is found in the upper body, lower body, upper extremities, lower extremities, ventral surface and dorsal surface of the subject to be inspected, but these abnormalities cannot be detected by the aforesaid conventional method. In the light of the foregoing situations, the present invention seeks to provide a biological information monitoring system capable of detecting biological information from at least one set of multiple positions of upper and lower bodies, upper and lower extremities, ventral and dorsal surfaces, complementary upper and lower parts and complementary front and back parts of the body of a subject so as to determine a physical abnormality at its earliest stage with higher accuracy than conventional methods.

[Patent Literature 1] Japanese Patent Application Publication HEI 10-155749(A) “System for monitoring and alerting human health condition”

[Patent Literature 2] Japanese Patent Application Publication No. 2000-93398(A) “Abnormal symptom detector and abnormality warning system”

[Patent Literature 3] Japanese Patent Application Publication HEI 6-242206(A) “Emergency relief system”

[Patent Literature 4] Japanese Patent Application Publication No. 2001-353130(A) “Information detector for acquiring physical information”

[Patent Literature 5] Japanese Patent Application Publication No. 2003-24287(A) “Device for monitoring physical condition”

[Patent Literature 5] PCT International Patent Publication No. WO2004/89202 “Biological information monitoring system”

### DISCLOSURE OF INVENTION

### Problems to be Solved by the Invention

A problem to be solved by the present invention is to provide a biological information monitoring system capable of determining a physical abnormality at its earliest stage with higher accuracy than conventional methods by detecting biological information at multiple positions of the body of a subject.

### Means of Solving the Problems

In order to solve the above problems according to the present invention, there is provided a biological information monitoring system comprising a plurality of biological information sensor modules to be attached to at least one set of positions of upper and lower bodies, upper and lower extremities, ventral and dorsal surfaces, complementary upper and lower parts and complementary front and back parts of the body of a subject, which biological information sensor modules each incorporate a biological information sensor for detecting the biological information and a wireless-enabled communication means capable of communicating the biological information, in which a determination means for determining an abnormality by comparing the biological information detected by the biological information sensor incorporated in one of the biological information sensor modules itself with another biological information received by the communication means in the other biological information sensor module.

### Effect of the Invention

According to the present invention, the biological information monitoring system capable of determining a physical abnormality at its earliest stage with higher accuracy than conventional methods by detecting biological information at multiple positions of the body of a subject can be realized.

### BRIEF DESCRIPTION OF THE DRAWINGS

FIG 1 Exploded perspective view showing a biological information sensor module in one embodiment of the present invention.

FIG 2 Perspective view showing the biological information sensor module of FIG. 1.

FIG 3 Front view of FIG. 2.

FIG 4 Bottom view of FIG. 2.

FIG 5 Criteria for determination of predictive detection of economy-class syndrome.

FIG 6 Criteria for determination of physical abnormality detection of general adult males in brisk walking (body surface temperature).

FIG 7 Criteria for determination of physical abnormality detection of general adult males in brisk walking (breath sound).

FIG 8 Diagrammatical view of a biological information monitoring system incorporating the biological information sensor of FIG. 1.

FIG 9 Flow chart schematically showing the biological information monitoring system of FIG. 8.

### EXPLANATION OF REFERENCES NUMERALS

1, 1A and 1B Sensor modules

2 Outer case

3 Battery

4 Main substrate

5 Sensor substrate

6 Double-sided adhesive tape

6a Apertures

7 Reset switch

8 External temperature sensor

9 Barometric sensor

10 Humidity sensor

11 Chip antenna

12 tilting sensor

13 Battery holder

13a and 13b Electrodes

14 Electromagnetic shielding plate

15 permeable protective cover

15a Apertures

16 Temperature sensor

17 Waterproof microphone serving as a pulse rate meter

18 Blood-pressure/pulse sensor

19 Thermal insulator

20 Main integrated circuit

21 Integrated circuit for wireless communication

22 Vibrator serving as warning means

28 Flexible joint

29 Host computer

S1 Step for attaching biological information sensors to the subject body

S2 Step for measuring biological information

S3 Step for measuring surrounding circumstances

S4 Step for storing biological information and surrounding circumstances in a memory

S5 Step for determining whether biological information falls within the normal range

S6 Step for wireless communication between biological information sensors 1A and 1B

S7 Step for calculating difference in biological information detected by biological information sensors 1A and 1B to determine whether the difference falls within the normal range

S8 Step for issuing a warning to a subject being inspected

S9 Step for notifying physical abnormality by wireless signals

S10 Step for identifying the subject being inspected with biological information sensors 1A and 1B by using identification signal (ID signal)

### BEST MODE FOR CARRYING OUT THE INVENTION

The biological information monitoring system according to the present invention is featured by comprising a plurality of biological information sensor modules to be attached to at least one set of positions of upper and lower bodies, upper and lower extremities, ventral and dorsal surfaces, complementary upper and lower parts and complementary front and back parts of the body of a subject, in which the biological information sensor modules each incorporate a biological information sensor for detecting the biological information and a wireless-enabled communication means capable of communicating the biological information, and a determination means for determining an abnormality by comparing the biological information detected by the biological information sensor incorporated in one of the biological information sensor modules itself with another biological information received by the communication means in the other biological information sensor module.

As the biological information detected by the biological information sensor, there can be enumerated body surface temperature, pulsebeat, blood pressure, myoelectric potential, sounds, image, positional information, physical motion, and inclination of a subject being inspected.

For example, the sensors may be attached to the both arms and both feet of the subject. Blood is fundamentally collected in a body part making an active movement consequently to increase the body surface temperature of the subject. The increased body surface temperature is gradually decreased after stopping the movement. However, if transition of the body surface temperature, which is expected to be relatively symmetric on the right and left sides of the body in a normal situation, are asymmetric, it is conceivable that the subject possibly has any abnormality on the one side of the subject's body. Otherwise, the abnormality of the body or acrocinesia of sports training workout can be appreciated by determining whether behavior of transition of the body surface temperature at the upper and lower positions of the subject's body from the elevated temperature to the lowered temperature deviates from the usual status of the subject.

By way of example, the changes in body surface temperature with respect to the arms and feet of the subject and the changes in myoelectric potential as the result of training workout can be observed before and after having worked out.

As another example, the measurement of breath sounds picked up respectively from both chest and back of the subject during exercise makes it possible to find out if a problem occurs in one of the respiratory organs. Further, the effect or acrocinesia of training workout and the physical condition before and after having worked out can be appreciated from the change in breath sound.

Furthermore, an economy-class syndrome (traveler's syndrome) can be predictively anticipated by observing a difference in body surface temperature, pulsebeat, blood pressure and/or myoelectric potential between the lower extremity and the upper extremity of the subject.

It is desirable to make an abnormality determination in such a manner that an abnormality is determined to occur when differences in body surface temperature, pulsebeat and blood pressure measured from at least one pair of bodily sites of the upper and lower parts of the body, upper and lower extremities, or ventral and back surfaces of the subject's body are 3 to 5° C. or more, 7 beats or more per minute, 10 mmHg or more, respectively.

The biological information sensor module may be provided with a warning means to issue a warning when a biological status to be observed is determined as abnormal by a determination means.

In at least one of the biological information sensor modules, there may be provided with a communication means for communicating the determination result from the determination means to the outside, and an electronic device outside of the biological information sensor module, which is capable of receiving the determination result given from the communication means.

Further in at least one of the biological information sensor modules, there may be provided with a memory for storing at least one of the determination result from the determination means and the biological information measured by the biological information sensor, thus to accumulate the measured biological information.

There may be used an electronic device for transmitting data to the biological information sensor modules by wireless communication, so that the abnormality determination can be made with reference to the data transmitted from the electronic device.

With the communication means for communicating with the outside, identification signals for identifying biologic subjects attached with the biological information sensor modules can be notified along with the determination result, so that the biologic subject having the determination result transmitted by the communication means can be identified referring to the identification signal sent with the determination result by reading the identification signal by the external electronic device.

The “wireless communication” referred to here is an inclusive term implying transmission and reception by wireless. In the concrete, this term includes interactive connection while executing error handling by wireless transmission and reception.

One embodiment of the present invention will be described hereinafter in detail.

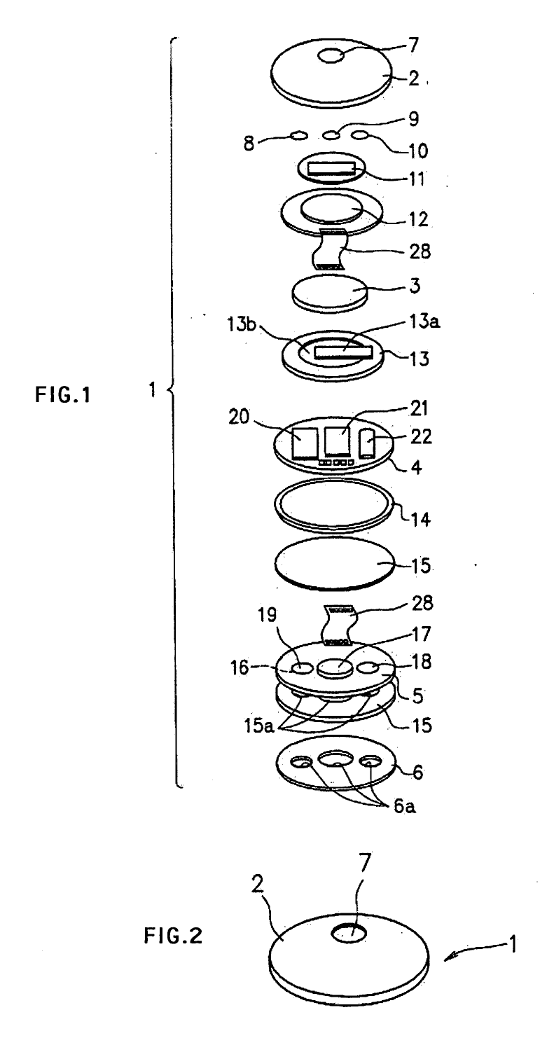

FIGS. 1 through 4 illustrate the biological information sensor module 1 in one embodiment of the biological information monitoring system according to the present invention. The biological information sensor module 1 is formed by incorporating a battery 3, a main substrate 4 and a sensor substrate 5 in an outer case 2 and provided on its bottom surface with a nonallergenic potency double-sided adhesive tape 6 having air permeability so as to be attached directly to the human body. To be more specific, the outer case 2 is formed of synthetic resin or the like in a small bowl shape opened to the lower face thereof (e.g. 37 mm in diameter and 7.2 mm in thickness), and provided on its upper part with a reset switch 7. Inside the outer case 2, there are mounted three environment sensors, that is, an ambient temperature sensor 8, a barometric sensor 9 and a humidity sensor 10. Further inside the outer case 2, there are mounted in order a chip antenna 11 for wireless communication, a tilting sensor 12, a battery 3 (button type lithium batter), a battery holder 13, a main substrate 4, an electromagnetic shielding plate 14, and a permeable protective cover 15. The sensor substrate 5 and permeable protective cover 15 serve to fixedly cover the outer case 2. The double-sided adhesive tape 6 having apertures 6a is stuck onto the outside surface of the permeable protective cover 15. The “inside” referred to here means the inner side (upper side in FIG. 1) of the outer case 2, and the “outside” referred to here means the bottom side (lower side in FIG. 1) opened in the outer case 2.

Each of the component parts constituting the aforementioned biological information sensor module 1 will be described. In this embodiment, on the sensor substrate 5, there are mounted three biological information sensors, i.e. temperature sensor 16, pulse rate meter 17 formed of a waterproof microphone, and blood-pressure/pulse sensor 18 (for instance, an optical sensor based on the technology disclosed in Japanese Patent Application Publication HEI 07-88090(A)).

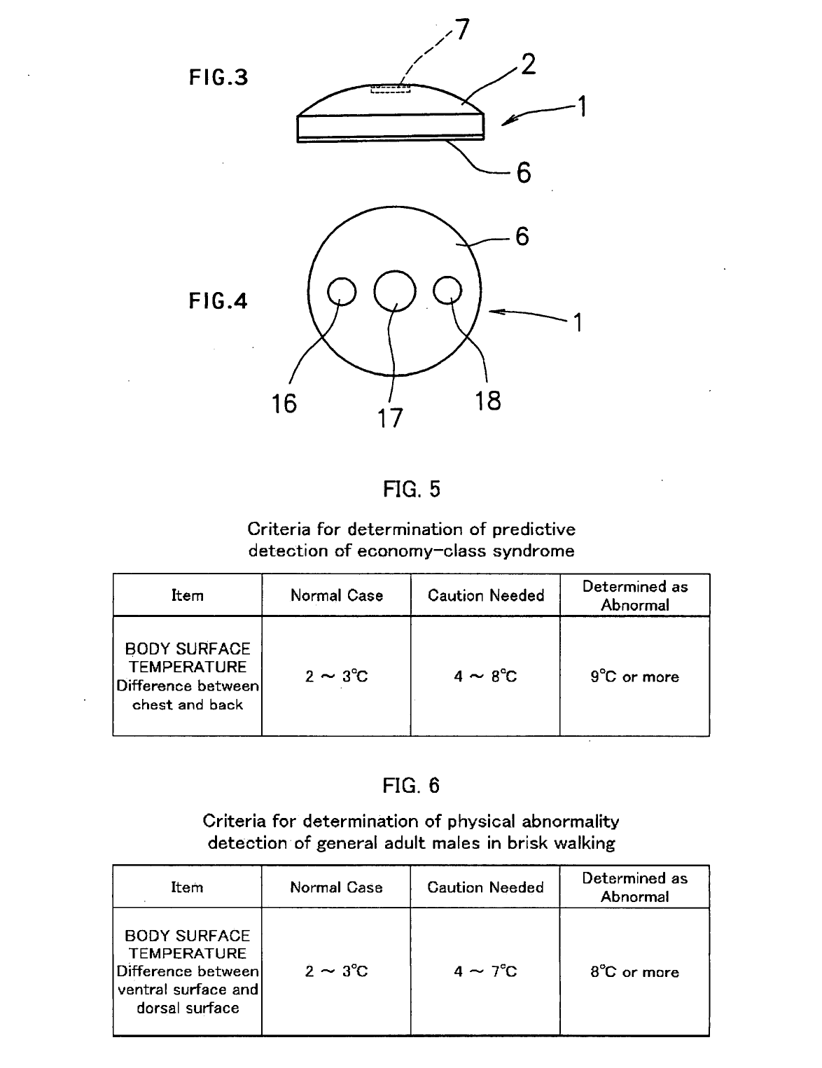

The detection parts of the biological information sensors (temperature sensor 16, pulse rate meter 17 and blood-pressure/pulse sensor 18) are exposed to the outside (lower side in FIG. 1) through not-shown opening apertures in the sensor substrate 5. The inside surface (upper surface in FIG. 1) of the temperature sensor 16 is sealed by a thermal insulator 19 to be thermally shielded from electronic elements or other heat sources on the main substrate 4. To the outside surface of the sensor substrate 5, the permeable protective cover 15 is attached, and further onto top of the outside surface thereof, the double-sided adhesive tape 6 is stuck, but the biological sensors (temperature sensor 16, pulse rate meter 17 and blood-pressure/pulse sensor 18) each have the detection part exposed to the outside through the apertures 15a in the permeable protective cover 15 and apertures 6a in the double-sided adhesive tape 6 (cf. FIG. 4).

The biological sensors are not limited to those described above, but may be made of a variety of sensors as known conventionally, and therefore, will not be described here in detail. The biological sensor may preferably be a small, light element capable of being operated with low power consumption for a long life by the battery 3 so as to detect the biological information with a high degree of accuracy.

On the main substrate 4, there are mounted a main integrated circuit 20 including a measurement calculating unit (determination means), a control unit (CPU) and a memory (storage means), an integrated circuit 21 for wireless communication, and a vibrator 22 serving as warning means. Onto the outside surface of the main substrate 4, a metal electromagnetic shielding plate 14 formed in a slightly concave shape, and the permeable protective cover 15 are fixed. The memory has a calendar function and a timer function for utilization in determining various abnormalities as described later.

The battery holder 13 serves as a protective cover and has a not-shown analogue circuit such as an amplifier mounted thereon. The battery holder 13 is provided with a pair of electrodes 13a and 13b coming in contact wit be electrodes of the battery 3. Inside the battery holder 13, the battery 3 of a button type is detachably placed.

The component elements set within the outer case 2, i.e. biological information sensors (temperature sensor 16, pulse rate meter 17 and blood-pressure/pulse sensor 18), main integrated circuit 20, integrated circuit 21 for wireless communication, vibrator 22, tilting sensor 12, chip antenna 11, three environment sensors (ambient temperature sensor 8, barometric sensor 9 and humidity sensor 10), and reset switch 7, are in electrical connection with one another on flexible joints 28 and the electrodes 13a and 13b of the battery holder 13 connected to the both electrodes of the battery 3. These component elements are supplied with an electric power from the battery 3 and controlled primarily by the CPU of the main integrated circuit 20 so as to perform writing and reading of data relative to the memory of the main integrated circuit 20. The electric circuits used herein may be composed of a variety of common circuits as known conventionally, and therefore, are not limited to those described above and will not be described here in detail. The components and kinds of the environmental sensors and biological information sensors are also not limited to those described above and may be modified and combined in various ways.

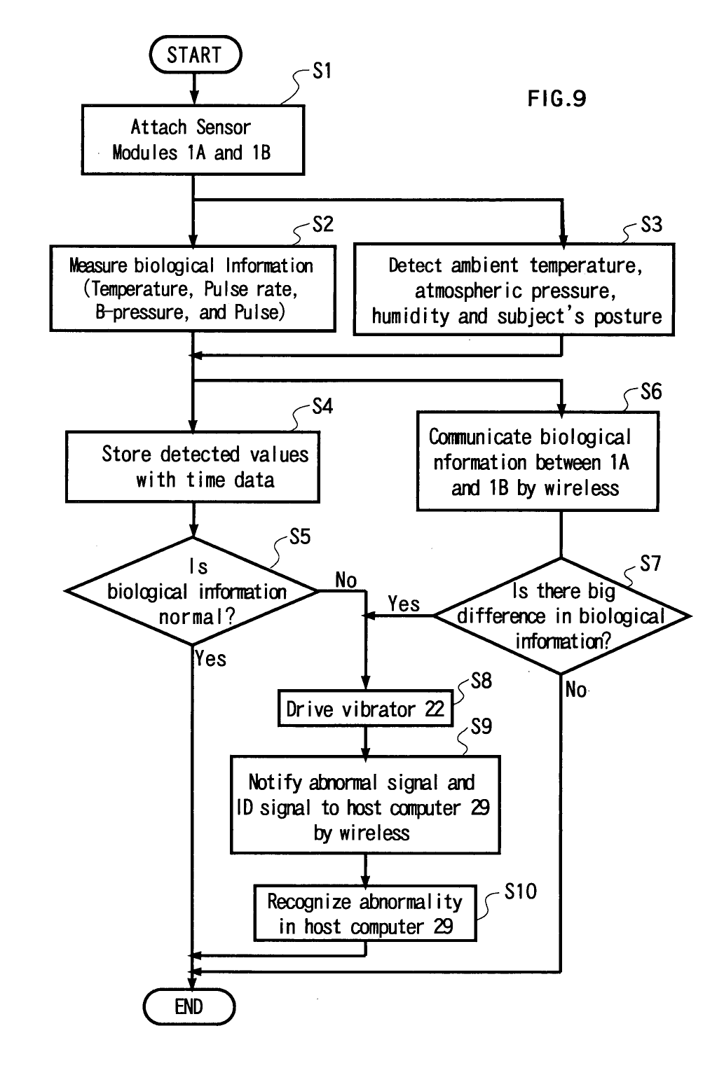

Next, the biological information monitoring system composed of the aforementioned biological information sensor modules 1 will be described hereinafter with reference to FIG. 8 and FIG. 9.

In this embodiment, two biological information sensor modules 1 as defined above (for descriptive purposes, one of the biological information sensor modules is denoted as “1A”, and the other biological information sensor module is denoted as “1B”) are attached to the subject with the double-sided adhesive tape 6 in such a state that the respective biological information sensors (temperature sensor 16, pulse rate meter 17 and blood-pressure/pulse sensor 18) have their detection parts coming in contact with at least one pair of relevant positions of upper and lower bodies, upper and lower extremities, ventral and dorsal surfaces, complementary upper and lower parts and complementary front and back parts of the body of a subject (for example, contrastive positions of the chest and back, the chest and abdomen, or the abdomen and femur). (Step S1) Then, electric power is supplied from the battery 3 to the sensor modules to measure the body surface temperature with the temperature sensor 16, the pulse rate with the pulse rate meter 17 and the blood pressure and pulsebeat with the blood-pressure/pulse sensor 18 under the control of the CPU of the main integrated circuit 20. (Step S2) Thus, while measuring the multiple kinds of biological information (body surface temperature, pulsebeat, blood pressure, myoelectric potential, etc.), the posture (standing or lying posture) of the subject being inspected is detected with the tilting sensor 12, an ambient temperature is measured with the ambient temperature sensor 8, an atmospheric pressure is measured with the barometric sensor 9, and humidity is measured with the humidity sensor 10. (Step S3)

The four kinds of biological information (body surface temperature, pulsebeat, blood pressure and myoelectric potential), which are measured with the temperature sensor 16, the pulse rate meter 17 and the blood-pressure/pulse sensor 18 and the ambient information (ambient temperature, atmospheric pressure and humidity), the posture data of the subject are stored in the memory of the main integrated circuit 20 together with measurement time or the time elapsed from starting of the monitoring (Step S4). Then, the measurement calculating unit makes a determination whether biological information falls within a normal range (Step S5). The range to be considered as normal for the biological information such as the body surface temperature is preferably determined individually for the standing state (active state) and the lying state (resting state) of the subject, so that a correction can be made according to the attitude of the tilting sensor 12.

Simultaneously, the wireless communication integrated circuit 21 using the chip antenna 11 communicates the measured biological information between the biological information sensor modules 1A and 1B by wireless (Step S6). In one of the biological information sensor modules, (e.g. module 1A), the biological information measured directly by the biological information sensors (temperature sensor 16, pulse rate meter 17 and blood-pressure/pulse sensor 18) incorporated in itself is compared with the biological information received by the wireless communication integrated circuit 21 via the chip antenna 11 and transmitted from the other biological information sensor module (e.g. module 1B). Then, the measurement calculating unit in the main integrated circuit 20 finds a difference between the measured values of biological information obtained by the both biological information sensor modules 1A and 1B, to determine whether the difference falls within the normal range (Step S7).

In Step S5 in this embodiment, in addition to the determination whether the difference of the biological information measured by the biological information sensors (temperature sensor 16, pulse rate meter 17 and blood-pressure/pulse sensor 18) falls within a normal range prescribed for the biological information detected by the biological information sensors, thereby to find out the presence or absence of physical abnormality, the presence or absence of physical abnormality is inspected by determining whether the difference of the biological information measured by the paired biological information sensor modules 1A and 1B attached to at least one pair of relevant positions of upper and lower bodies, upper and lower extremities, ventral and dorsal surfaces, complementary upper and lower parts and complementary front and back parts of the body of the subject falls within a normal range prescribed for the biological information detected by the sensor modules in Step S7. The advantage of the processing in these steps will be described later.

When the inspection results obtained in both Step S5 and Step S7 are determined as normal, the sequence of inspecting processes is finalized. However, if the inspection result obtained in one or both of Step S5 and Step S7 is determined as abnormal, the vibrator 22 is driven to vibrate by the CPU of the main integrated circuit 20, thus to allow the subject to perceive the abnormality (Step S8). Simultaneously, an abnormality signal showing the abnormality is issued from the wireless communication integrated circuit and sent from the chip antenna to the outside by wireless (Step S9). At this time, the identification signal (ID signal) for identifying the subject attached with the biological information sensor modules 1A and 1B is delivered along with the abnormality signal. Then, the host computer 29 placed distant from the subject being inspected receives the abnormality signal and the ID signal produced in Step S9, thus to recognize the physical abnormality of the subject attached with the biological information sensor modules 1A and 1B (Step S10). Thus, the sequence of inspecting processes is finalized. However, the aforementioned inspecting processes are sequentially repeated without a break to continuously monitor the biological information for a long time in practice.

In Step S8 of the embodiment of the invention, while the subject being inspected himself can recognize his physical abnormality by feeling the vibration generated by the vibrator 22, thus to take countermeasures to the abnormality promptly, a doctor or nurse handling the host computer 29 can also know the physical abnormality of the subject, thus to take countermeasures to the abnormality promptly.

According to the invention, since the biological information such as the body surface temperature and pulsebeat is detected from one set of positions of upper and lower bodies, upper and lower extremities, ventral and dorsal surfaces, complementary upper and lower parts and complementary front and back parts of the body of the subject, it is highly possible to recognize the abnormality even when a change in biological information occurs only in a part of the body of the subject due to the physical abnormality. Besides, since the abnormality is recognized on the basis of the difference in biological information detected from one set of positions of upper and lower bodies, upper and lower extremities, ventral and dorsal surfaces, complementary upper and lower parts and complementary front and back parts of the body of the subject, the present invention enables early detection and preservation of the abnormality, which have been hardly made with reference to mere biological information obtained by a conventional method.

Further according to the invention, by detecting the difference in body surface temperature from at least one set of positions of upper and lower bodies, upper and lower extremities, ventral and dorsal surfaces, complementary upper and lower parts and complementary front and back parts of the body of the subject, extraordinary states of the subject being inspected can be recognized promptly before the subject is aware of the abnormality.

For instance, in a case of thrombosis crisis in economy-class syndrome, it is possible to know a foretaste of growth in thrombus from a sign of swelling of leg due to continuous sitting.

### [First Practical Application] <Economy-Class Syndrome>

As the first practical application of the invention, appreciation of the foretaste of economy-class syndrome can be made. That is, the foretaste of the economy-class syndrome (traveler's syndrome) can be appreciated from deviation of the body surface temperatures detected from the lower and upper extremities of the subject's body from the usual normal temperatures and, pro re nata, differences in pulse beat, blood pressure and myoelectric potential. The positions to which the sensor modules are attached are shown in FIG. 8, and one example of criteria for determination of physical abnormality is shown in FIG. 5.

### [Second Practical Application] <Detection of Physical Abnormality and Acrocinesia: Recurrence of Body Surface Temperature and Breath Sound>

As the second practical application of the invention, appreciation of the physical abnormality and acrocinesia can be made in the field of sports medicine. The positions to which the sensor modules are attached are shown in FIG. 8, and one example of criteria for determination of physical abnormality is shown in FIG. 6.

There is made an assumption that the sensors are attached to the both arms and feet of the subject being inspected by way of example. Blood is fundamentally collected in a body part making an active movement consequently to increase the body surface temperature of the subject. The increased body surface temperature is gradually decreased after stopping the movement. However, if transition of the body surface temperature, which is expected to be relatively symmetric on the right and left sides of the body in a normal situation, are asymmetric, it is conceivable that the subject possibly has any abnormality on the one side of the subject's body. Otherwise, the abnormality of the body or acrocinesia of sports training workout can be appreciated by determining whether behavior of transition of the body surface temperature at the upper and lower positions of the subject's body from the elevated temperature to the lowered temperature deviates from the usual status of the subject.

As further example, the measurement of breath sounds picked up respectively from both chest and back of the subject during exercise makes it possible to find out if a problem occurs in one of the respiratory organs. Besides, acrocinesia of training workout and physical conditions before and after having worked out can be appreciated from the change in breath sound.

### [Third Practical Application] <Effect of Training Workout>

As the third practical application of the invention, appreciation of the effect of training workout can be made in the field of sports medicine. It is possible to observe the changes in body surface temperature with respect to the arms and feet of the subject and the changes in myoelectric potential as the result of training workout when exercising in the same manner before and after having worked out.

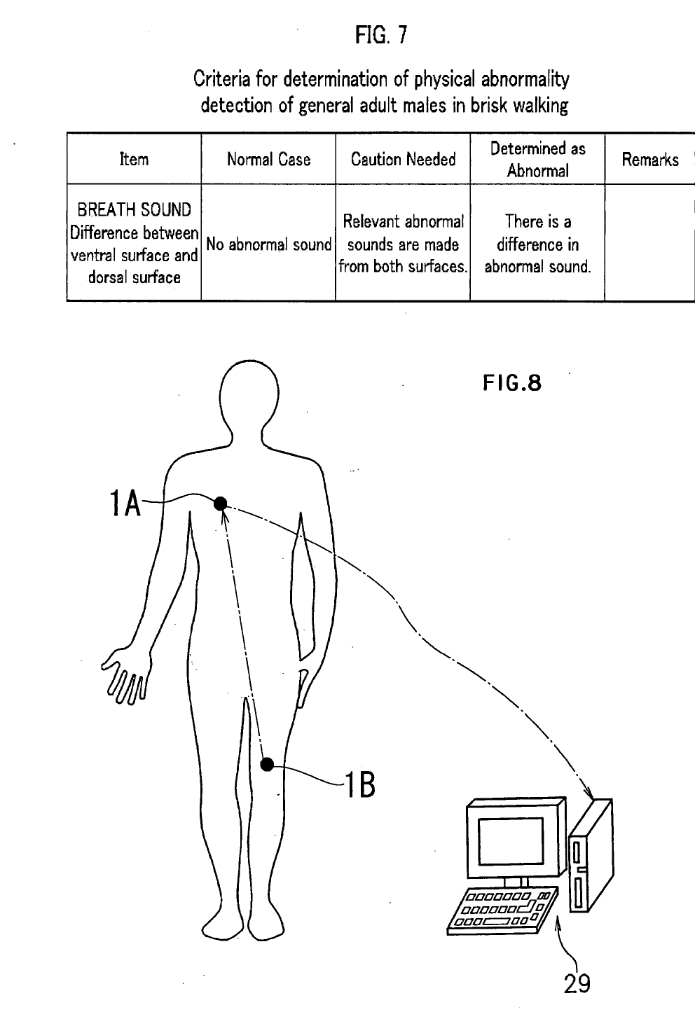

Alternatively, by measuring breath sounds picked up respectively from both chest and back of the subject during exercise to fine the change in breath sound or myoelectric potential before and after having worked out, the effect of training workout can be appreciated. One example of criteria for determination of physical abnormality is shown in FIG. 7.

### [Fourth Practical Application] <Acrocinesia of Sports>

Determination whether or not the body during running is in an abnormal state beyond the physical limits can be made by using the sensor modules attached to the both sides of the chest, the both sides of the back and the both thighs of the subject to be inspected.

### [Fifth Practical Application] <Rehabilitation>

An effect of rehabilitation can be observed by placing the sensor modules onto the both sides of the chest, the both sides of the back and the both thighs of the subject physically challenged due to a traffic accident or a patient suffering a stroke and continuing measurement of the biological information for a few days to several tens of days or more. If previous data before encountering an accident or getting a disease are accumulated, the effect of rehabilitation can be perceived.

### [Sixth Practical Application] <Security>

As an example of application for security, individuals entering a room can be authenticated by previously attaching the sensor modules to the both sides of the chest, the both sides of the back and the both thighs of the subjects to be supervised and collating record data, which have been accumulated in a database upon grasping individual working patterns shortly before entering the room.

### [Seventh Practical Application] <Baby>

Determination of the presence or absence of abnormalities in the brain and body of a baby can be made by collating data detected by the sensor modules attached to the both sides of the chest, the both sides of the back and the both thighs of the baby with standard values in a database.

### [Eighth Practical Application] <Dieting>

Determination of overdieting can be made by collating data detected by the sensor modules attached to the both sides of the chest, the both sides of the back and the both thighs of the subject being inspected with standard values in a database.

### [Ninth Practical Application] <Traffic Accident>

Determination of the presence or absence of abnormalities in the brain and body of a subject injured in a traffic accident can be made by collating data detected by the sensor modules attached to the both sides of the chest, the both sides of the back and the both thighs of the subject with activity data and biological information of a healthy subject.

### [Tenth Practical Application] <Industrials: Development of Humanoid Robot>

Measurement of working aspect of a humanoid robot can be made by using the sensor modules mounted onto the both sides of the chest, the both sides of the back and the both thighs of the humanoid robot. As the sensors used therein, there are a tilting sensor, a GPS sensor and an image sensor.

### [Eleventh Practical Application] <Muscular Dystrophy>

Therapeutic progresses of treatment, rehabilitation and disease state of muscular dystrophy can be recognized by measuring myoelectric potentials with the sensor modules attached to four or more symmetric positions in the upper and lower body and the right and left body on the half side of the patient's body, which displays the symptoms of muscular dystrophy, and the other half side free of the symptoms of muscular dystrophy and comparing data measured at the upper and lower positions of the body with data measured at the right and left positions of the patient's body.

### [Twelfth Practical Application] <Muscle Fatigue>

An effect in relieving fatigue can be perceived by comparing differences of values obtained by measuring myoelectric potential after exertion by using the sensor modules attached to four or more symmetric positions in the upper and lower body and the right and left body on the half side of a subject being inspected.

### [Thirteenth Practical Application] <Data Mining>

Data mining can be performed by accumulating, in a data server, value data measured by using the sensor modules including sensors for detecting biological information, myoelectric potential and tilting posture of a subject being inspected, which are permanently attached to the body of the subject, and comparing the measured value data with normal value data or past disease data in the data server or comparing the measured value with value data accumulated so far from the subject. This is applicable for detection of indication of pathogenesis, development in therapy, and discovery of side-effect of medication.

### [Fourteenth Practical Application] <Physical Abnormality>

By attaching the sensor modules to the body of a subject to measure triaxial biological data of the upper and lower parts, right and left parts and front and back parts (ventral and dorsal surfaces) at the positions denoted by 1A and 1B in the accompanying drawing by way of example for the purpose of medical examination the field of sports medicine or disease, there can be made left-right comparison in difference between the upper and lower parts (i.e. abnormality detection on the left and right halves of the body), front-back comparison in difference between the upper and lower parts (i.e. abnormality detection on anteroposterior axis), left-right comparison in difference of the front and back parts (i.e. abnormality detection on the left and right halves of the body), and upper-lower comparison in difference of the front and back parts (i.e. abnormality detection on anteroposterior axis).

### [Fifteenth Practical Application] <Physical Abnormality of Solitary Old People>

Motion characteristics of solitary old people can be analyzed by attaching the sensor modules onto the positions denoted by 1A and 1B in the accompanying drawing by way of example for the purpose of analyzing the motion characteristics, to confirm the safety of the solitary old people.

[Sixteenth Practical Application] <Physical Abnormality under Dangerous Environment>

Motion characteristics of a subject being inspected at work under a high- or cool-temperature environment or dangerous environment of producing poisonous gases can be analyzed by attaching the sensor modules onto the positions denoted by 1A and 1B in the accompanying drawing by way of example for the purpose of analyzing the motion characteristics, to confirm the safety of the subject.

### INDUSTRIAL APPLICABILITY

Since the present invention can provide the biological information monitoring system capable of detecting biological information from at least one set of multiple positions of upper and lower bodies, upper and lower extremities, ventral and dorsal surfaces, complementary upper and lower parts and complementary front and back parts of the body of a subject being inspected, it becomes possible to determine a physical abnormality with ease at its earliest stage, which could not be detected by a conventional method, so that the safety of the subject being inspected can be enhanced dramatically.

Further according to the invention, appreciation of the foretaste of economy-class syndrome can be made and acrocinesia of sports training workout can be appreciated by quantifying the effect of training workout in the field of sports medicine.

1-11. (canceled)

12. A biological information monitoring system comprising a plurality of biological information sensor modules, at least one of said biological information sensors being attached to a relatively upper part of a body and at least one of said biological information sensors being attached to a relatively lower part of the body or at least one of said biological information sensors being attached to the ventral surface of the body and at least one of said biological information sensors being attached to the dorsal surface of the body, said biological information sensor modules each having a biological information sensor for detecting the biological information and a wireless-enabled communication means capable of communicating the biological information, wherein a determination means for determining an abnormality by comparing the biological information detected by said biological information sensor incorporated in one of said biological information sensor modules with another biological information received by said communication means in the other biological information sensor module.

13. The biological information monitoring system claimed in claim 12, wherein said biological information detected by said biological information sensor is at least one of information of body surface temperature, pulse, blood pressure, myoelectric potential, sounds, image, positional information, physical motion, and inclination.

14. The biological information monitoring system claimed in claim 12, wherein said biological information sensor is a temperature sensor, and said determination means serves to make an abnormality determination when a difference between the maximum detection temperature and the minimum detection temperature of said temperature sensor is 3 to 5° C. or more.

15. The biological information monitoring system claimed in claim 12, wherein said biological information sensor is a pulse sensor, and said determination means serves to make an abnormality determination when a difference between the maximum detection pulse rate and the minimum detection pulse rate of said pulse sensor is 7 beats or more per minute.

16. The biological information monitoring system claimed in claim 12, wherein said biological information sensor is a blood-pressure sensor, and said determination means serves to make an abnormality determination when a difference between the maximum detection blood pressure and the minimum detection blood pressure of said blood-pressure sensor is 10 mmHg or more.

17. The biological information monitoring system claimed in claim 12, wherein said biological information sensor module has a warning means to issue a warning when said determination means detects an abnormality.

18. The biological information monitoring system claimed in claim 12, wherein at least one of said plurality of biological information sensor modules has a communication means capable of communicating to the outside the determination result from said determination means, and further comprising an external electronic device disposed outside said biological information sensor modules and capable of receiving the determination result notified from said communication means.

19. The biological information monitoring system claimed in claim 12, wherein at least one of said plurality of biological information sensor modules has a memory for storing at least one of the determination result from said determination means and the biological information measured by said biological information sensor.

20. The biological information monitoring system claimed in claim 12, further comprising an electronic device for transmitting data to said biological information sensor modules by wireless communication, wherein said determination means makes abnormality determination referring to said data transmitted from said electronic device.

21. The biological information monitoring system claimed in claim 18, featured in that said communication means for communicating with the outside serves to notify an identification signal identified with respect of each biological body attached with said biological information sensor modules along with the determination result, and said external electronic device serves to read said identification signal with said determination result to identify the living body issuing said determination result.

22. The biological information monitoring system claimed in claim 12, featured by determining physical abnormality, a relationship between the physical abnormality and external environment or evaluation of a motion effect, or analyzing motion characteristics by data mining.

23. A system for anticipating prediction of deep vein thrombosis or acute pulmonary thromboembolism, comprising a plurality of biological information sensor modules, at least one of said biological information sensors being attached to a relatively upper part of a body and at least one of said biological information sensors being attached to a relatively lower part of the body or at least one of said biological information sensors being attached to the ventral surface of the body and at least one of said biological information sensors being attached to the dorsal surface of the body, said biological information sensor modules each having a biological information sensor for detecting the biological information and a wireless-enabled communication means capable of communicating the biological information, wherein a determination means for determining an abnormality by comparing the biological information detected by said biological information sensor in one of said biological information sensor modules with biological information received by said communication means in the other biological information sensor modules, and said biological information sensor is a temperature sensor, and said determination means serves to make an abnormality determination when a difference between the maximum detection temperature and the minimum detection temperature of said temperature sensor is 3 to 5° C. or more.

24. A system for anticipating prediction of deep vein thrombosis or acute pulmonary thromboembolism, comprising a plurality of biological information sensor modules, at least one of said biological information sensors being attached to a relatively upper part of a body and at least one of said biological information sensors being attached to a relatively lower part of the body or at least one of said biological information sensors being attached to the ventral surface of the body and at least one of said biological information sensors being attached to the dorsal surface of the body, said biological information sensor modules each having a biological information sensor for detecting the biological information and a wireless-enabled communication means capable of communicating the biological information, wherein a determination means for determining an abnormality by comparing the biological information detected by said biological information sensor in one of said biological information sensor modules with biological information received by said communication means in the other biological information sensor modules, and said biological information sensor is a pulse sensor, and said determination means serves to make an abnormality determination when a difference between the maximum detection pulse rate and the minimum detection pulse rate of said pulse sensor is 7 beats or more per minute.

25. A system for anticipating prediction of deep vein thrombosis or acute pulmonary thromboembolism, comprising a plurality of biological information sensor modules, at least one of said biological information sensors being attached to a relatively upper part of a body and at least one of said biological information sensors being attached to a relatively lower part of the body or at least one of said biological information sensors being attached to the ventral surface of the body and at least one of said biological information sensors being attached to the dorsal surface of the body, said biological information sensor modules each having a biological information sensor for detecting the biological information and a wireless-enabled communication means capable of communicating the biological information, wherein a determination means for determining an abnormality by comparing the biological information detected by said biological information sensor in one of said biological information sensor modules with biological information received by said communication means in the other biological information sensor modules, and said biological information sensor is a blood-pressure sensor, and said determination means serves to make an abnormality determination when a difference between the maximum detection blood pressure and the minimum detection blood pressure of said blood-pressure sensor is 10 mmHg or more.
